(19) United States
(12) Patent Application Publication
(10) Pub. No.: US 2005/0240211 A1
(43) Pub. Date: Oct. 27, 2005

(54) Apparatus and method for selectably treating a fallopian tube

(76) Inventors: Stefan Sporri, Berne (CH); Brent Bell, Dickinson, TX (US); Eric K. Chan, Missouri City, TX (US); Ashok Gowda, Houston, TX (US)

Correspondence Address: Elizabeth R. Hall, 1722 Maryland Street, Houston, TX, 77006-1716, US

(21) Appl. No.: 11/088,042

(22) Filed: Mar. 23, 2005

Related U.S. Application Data
(60) Provisional application No. 60/563,995, filed on Apr. 21, 2004.

(51) Int. Cl. A61M 29/00

(52) U.S. Cl. 606/193

(57) ABSTRACT

The present invention is an apparatus and methods for selectably assessing a fallopian tube. The apparatus is a device that allows for treatment and/or the assessment of the patency of each fallopian tube independently by isolating at least one cornual regions from the uterine cavity. The method of the present invention is based on use of the device and allows for assessment of fallopian tube patency without the use of dyes of x-rays.

### CROSS REFERENCE TO RELATED APPLICATIONS

This application claims priority to U.S. Provisional Patent Application Ser. No. 60/563,995 filed Apr. 21, 2004 and entitled “Apparatus and Method for Selectably Assessing a Fallopian Tube” by inventors Stefan Sporri, et al.

### BACKGROUND OF THE INVENTION

### FIELD OF THE INVENTION

The invention relates to an apparatus (device) and method for selectably treating the right or the left fallopian tube. More particularly, the present invention relates to a catheter device for infusion and maintenance of fluid selectively on either the right or left side of a patient's uterus.

### DESCRIPTION OF THE RELATED ART

Infertility is a common reproductive disorder among couples of childbearing age. Current estimates suggest that up to 6 million couples in the U.S. alone are affected by infertility. Ordinarily these couples will undergo a series of diagnostic evaluations to isolate the most probable cause of their infertility. The initial tests to determine if the infertility is associated with the female begin with assessment of fallopian tube patency. The fallopian tubes are the organs in which conception occurs and thus, at least one fallopian tube must be open to permit the passage of sperm to the ovum. A number of conditions, including the presence of inflammatory disease, fibrotic lesions, cellular debris and mucous can result in occlusion or blockage of the fallopian tubes. Assessment of the patency of a patient's fallopian tubes is accomplished by the procedures described below. The devices used for these procedures include a catheter to introduce the fluid into the uterus, as well as a means for sealing off the bottom of the uterus or the cervical canal to prevent backflow of the fluid into the vaginal canal. Such devices are described in U.S. Pat. Nos. 5,624,399; 5,935,098; 5,540,658; 5,372,584; 5,259,836; 5,248,304; 4,611,602; 4,601,698; 4,430,076; 4,089,337; 3,721,229; and 2,441,237.

In order to determine whether the fallopian tubes are patent or occluded, a physician will initially perform a diagnostic procedure called hysterosalpingography (HSG). HSG involves the injection of radiographic contrast medium (or dye) transcervically into the uterus to allow the physician to observe and evaluate the flow of dye through the fallopian tubes under fluoroscopy (X-ray). This procedure can be painful and is also known to be highly inaccurate due to the potential of fallopian tube spasms. Frequent tubal spasms, a natural function and reaction of the tubes, may temporarily close the tube, preventing flow and resulting in a false diagnosis of occlusion. The likelihood of a false diagnosis is increased by the fact that visualization time is minimized in order to prevent prolonged exposure to X-rays and its associated complications.

When HSG indicates a tubal occlusion, the physician will perform another diagnostic confirmatory procedure called Selective Hysterosalpingography (SHSG). SHSG involves selectively catheterizing the opening of the occluded fallopian tube and injecting radiographic contrast fluid at high pressures directly into the tubal lumen. The higher pressures used in SHSG will sometimes cause tubal spasms and/or debris to yield to the pressurized fluids whereby confirming whether the blockage is of legitimate or false concern. Occasionally, the injected fluids will result in reopening of the tube.

If SHSG fails to open the occluded fallopian tube the physician will perform yet another procedure called Transcervical Tubal Catheterization (TTC). TTC involves carefully feeding a platinum tipped guidewire into the tube as far as safely possible to mechanically unblock and recannulate the occluded lumen. Both SHSG and TTC serve dual purposes as both diagnostic and therapeutic tools for fertility assessment and treatment, respectively.

If couples fail to conceive after HSG, SHSG, and TTC, physicians will schedule a more invasive procedure called laparoscopic chromopertubation, more commonly known as “lap and dye”. This procedure is similar to SHSG but involves selective fallopian tube injection of colored dye while viewing the absence or presence of flow through the distal end of the fallopian tube using an endoscope positioned in the abdomen. In addition to direct visualization of the exiting dye, the endoscopic procedure serves also as an exploratory procedure to investigate and document any abnormalities found within the patient's reproductive system.

Aside from diagnosis of problems related to infertility, assessment of fallopian tube patency can also be used to confirm closure after tubal sterilization procedures. Currently, the most popular methods for tubal sterilization include the placement of a ring, clip, or electro-cauterization of the tube via laparoscopy. Although tubal closure in these procedures can normally be assessed at the time of surgery, newer non-surgical methods of sterilization are being developed which will require a means for confirming closure. These non-surgical methods are introduced via a transcervical approach by means of a hysteroscope and include mechanical devices, chemical sclerosing agents, and thermal methods. Such approaches will require the physician to determine if the procedure was successful and remains effective at regular intervals after the procedure.

Thus, there exists a need for an inexpensive, easy to perform, reliable method of assessing the patency of the fallopian tubes, which avoids exposure of the patient to harmful x-rays, eliminates mechanically inserting anything into the fallopian tube itself, and can be performed without the need for incisional surgery.

Additionally, there is a need for a means to selectively flush fallopian tubes for the purpose of clearing tubal obstructions using various flushing or medicated mediums. Similarly, there is a need for a means of assessing each fallopian tube's patency independent of the other tube.

### SUMMARY OF THE INVENTION

The invention contemplates a simple, inexpensive device for effectively and selectively determining the patency and/or treating each fallopian tube, independently. Two general embodiments of the present invention provide either (a) for selecting and sealing one fallopian tube, or (b) sealing both fallopian tubes and providing a means for selectively assessing or treating one fallopian tube at a time.

One general embodiment of the device includes a housing element with an inflatable element just proximal to the distal end of the housing element. The inflatable element contains two separate outlet orifices for injection of fluid on opposing sides of the inflatable element. The device can be vaginally inserted to extend through the cervix and into the uterus of the patient. Once inserted, the inflatable element is inflated within the uterus to effectively separate the two sides of the uterus and seal the cervical canal. Fluid is injected through either orifice in the inflatable element to fill the void region created by the inflatable element and the cornual region of each side of the uterus. Pressure within this region is monitored externally using a pressure monitoring source. If the fallopian tube being tested for patency is open, the pressure within the void region will fall due to fluid flowing through the fallopian tube and into the peritoneal cavity. However, if the pressure within the region being tested is maintained for an extended period, then the fallopian tube being examined is considered to be closed or occluded. The design of the device of the instant invention allows patency testing to be performed on each side of the patient selectively to determine the status of each fallopian tube independent of the other.

The present invention is therefore a device or apparatus for assessing patency of fallopian tubes in animals and humans. Specifically, the apparatus comprises a flexible housing element having a distal end and a proximal end; an inflatable member located on the distal end of said housing element; two injection lumens traversing the length of the housing element, wherein a first injection lumen terminates on a one side of said inflatable member, and a second injection lumen terminates on the opposite side of said inflatable member; a fluid supply (either a liquid or gas), is connected to the proximal end of said injection lumens; and a means for monitoring pressure within a fallopian tube, said pressure monitoring means being in communication with said injection means and said fluid supply.

In an alternative embodiment, the apparatus of the present invention includes two inflatable members, a right inflatable member and a left inflatable member, which are used to create two non-communicating regions within the uterus of the patient. In yet another alternative embodiment, the inflatable member is positioned at the tip of the housing element. In another alternative embodiment, the inflatable members, when inflated, form two cavities at the sites to which the first and second lumens exit the inflatable member. These cavities are concave regions in the outer surface of the inflatable member.

The present invention is also a method for assessing patency of fallopian tubes in a patient which comprises first inserting the apparatus of the instant invention into the uterus of the patient, inflating the inflatable member of the present invention, measuring a first pressure within the first and second lumens of the apparatus, injecting a fluid (either a liquid or gas) through the first and second lumens of the apparatus, and monitoring changes in pressure within said lumens so that patency of the fallopian tubes can be determined. The foregoing has outlined rather broadly several aspects of the present invention in order that the detailed description of the invention that follows may be better understood and thus is not intended to narrow or limit in any manner the appended claims which define the invention. Additional features and advantages of the invention will be described hereinafter which form the subject of the claims of the invention. It should be appreciated by those skilled in the art that the conception and the specific embodiment disclosed may be readily utilized as a basis for modifying or designing of the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1A is a schematic presentation of a portion of a female reproductive system including the cervix, uterus, and fallopian tubes and a cross sectional view of one embodiment of the patency assessment device embodying the principles of the present invention;

FIG. 1B is a close-up of the distal end of the patency assessment device shown in FIG. 1A;

FIG. 2 illustrates another embodiment of the patency assessment device;

FIG. 3 is a cross sectional view of one embodiment of the patency assessment device embodying the principles of the present invention deployed in a female uterus;

FIGS. 4A and 4B is a cross sectional view of the distal portion of one embodiment of the patency assessment device according to the principles of the present invention prior to deployment or placement into the uterus and after deployment or placement into the uterus;

FIGS. 5A and 5B illustrate a cross sectional view of an alternative embodiment of the distal portion of the patency assessment device;

FIG. 6A illustrates an alternative embodiment of the inflatable member located on the distal end of the patency assessment device of FIG. 3;

FIG. 6B illustrates an alternative embodiment of the patency assessment device;

FIGS. 7A and 7B are cross-sectional views of alternative embodiments of the patency assessment device having two opposed inflatable members;

FIGS. 8A-C illustrate an alternative embodiment of the patency assessment device as the device is being inserted, as the device is situated for testing a first fallopian tube, and as the device is situated for testing the contralateral fallopian tube;

FIG. 9 illustrates an alternative embodiment of the proximal portion of the patency assessment device;

FIG. 10 illustrates an alternative embodiment of the distal portion of the patency assessment device having an injection lumen surrounded by a vacuum line;

FIG. 11 illustrates an alternative embodiment of the distal portion of the patency assessment device shown in FIG. 7B but with a vacuum line similar to that shown in FIG. 10; and

FIG. 12 illustrates an alternative embodiment of the distal portion of the patency assessment device shown in FIG. 3B having a vacuum line similar to that shown in FIG. 10.

It is noted, however, that the drawings illustrate preferred embodiments of the invention and are therefore not to be considered limiting of its scope, for the invention may have equivalent embodiments.

### DESCRIPTION OF THE PREFERRED EMBODIMENTS

The present invention includes a device that can be vaginally inserted into the uterus of a female patient to inject fluids (i.e., liquids or gases) within a locally sealed region of the uterus for treating the fallopian tube or assessing its patency. The present invention also includes methods of use of the device for assessing fallopian tube patency and for injection of fluids into the fallopian tubes of a patient. In the context of the present invention, the patients contemplated to be subject to use of the methods would include humans as well as animals.

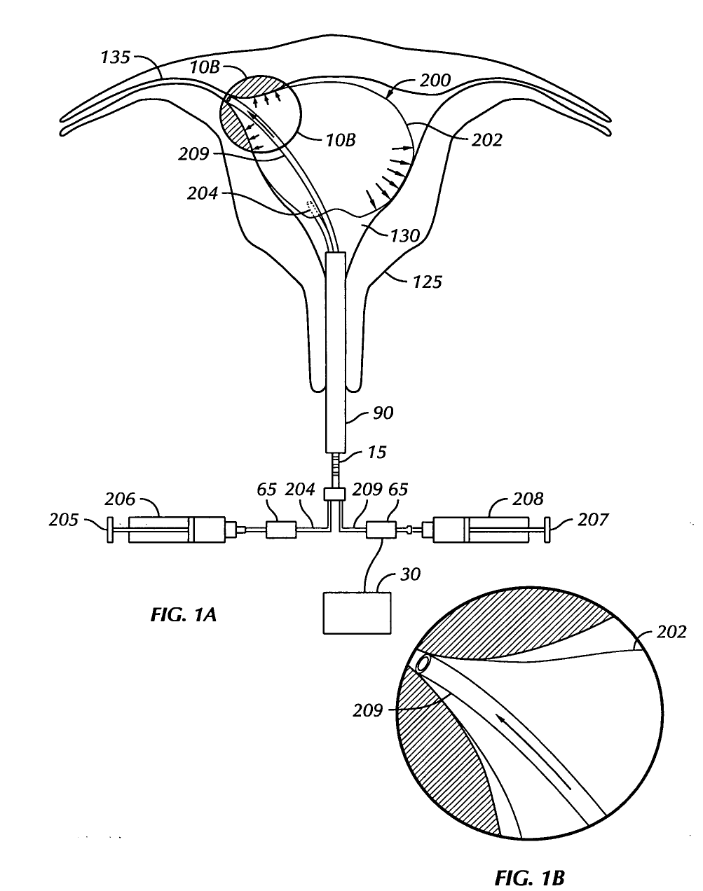

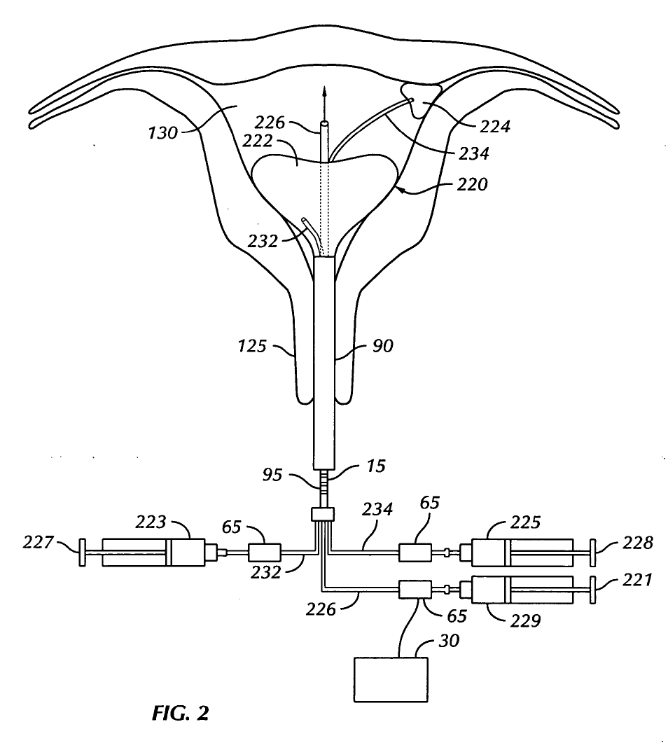

Referring now to the drawings, and initially to FIG. 1, it is pointed out that like reference characters designate like or similar parts throughout the drawings. The figures, or drawings, are not intended to be to scale. For example, purely for the sake of greater clarity in the drawings, wall thickness and spacing are not dimensioned as they actually exist in the assembled embodiment.

FIG. 1 illustrates some of the major elements of the female reproductive system including the uterus 100, fallopian tubes 135, and ovary 110. The lower part of the uterus 100 is integral with a vagina 115. The vagina 115 has a vaginal cavity 120 with the distal end of the vaginal cavity 120 being in communication with a cervix 125. The cervix 125 has a normally closed opening 127 and has a passage from the vaginal cavity 120 to the uterine cavity 130. The uterine cavity 130 has a top wall or fundus 132 that further includes side walls 133 which communicate with the cervix 125. The uterine cavity 130 varies as to size and configuration and is generally somewhat triangular in shape. The fallopian tubes 135 are paired hollow seromuscular organs that connect the uterus 100 to the two ovaries 110. The tubal ostium 140 is the entry point to the fallopian tube 135 from the uterus 100. The fimbria 145 is the distal end of the fallopian tubes 135 that opens to the peritoneal cavity.

A. Selecting and Sealing One Fallopian Tube at a Time

A first embodiment of the present invention that selects and seals one fallopian tube at a time is shown in FIGS. 1A and 1B.

FIG. 1A shows one embodiment of the patency assessment device 200 of the present invention vaginally inserted into and extending through the cervix 125 into the uterine cavity 130. Among the major components of the patency assessment device 200 is an elongated flexible housing element 15, a retractable sleeve 90, an inflatable member 202, an inflation tube 204, an inflation fluid supply 206 in fluid communication with the inflatable member 202 through the inflation tube 204, an injection fluid supply 208, an injection lumen 209, and a pressure monitoring system 30 located between the housing element 15 and the injection fluid supply 208.

The housing element 15 is used to maneuver the inflatable member 202 through the cervix 125 to the uterine cavity 130. The housing element 15 may be made of any rigid, semi-rigid, or flexible material, such as polyurethane, polyethylene, polyimide, or other polymers, plastics or metals. The housing element may be either internally and/or externally coated with specialized friction reducing hydrophilic or hydrophobic materials. Examples of such materials are Teflon from Dupont Chemical Company or Lubrilast from AST Products, Inc.

The length of the housing element 15 must be adequate to extend through the patient's vagina 115 and cervix 125 to reach the uterine cavity 130, as well have a sufficient length to conveniently connect to the inflation and injection fluid supplies 206 and 208. The length of the housing element 15 of the patency assessment device 200 to be used on humans would typically have a length ranging from about 10 cm to about 30 cm in length.

The size of the outside diameter of the housing element 15 requires one to consider the general diameter of an instrument that can be inserted through the cervical canal in a comfortable and non-traumatic fashion. The patency assessment device for use in humans would generally have an outside diameter that ranges from 2 mm to about 1 cm, and would preferably be approximately 4 mm.

A retractable sleeve 90 is positioned over the distal end of the housing element 15 such that during insertion the retractable sleeve 90 covers the deflated inflatable member 202. Markings on the housing element 15 assist in the proper orientation of the housing element 15. Once the patency assessment device 200 is positioned in the uterus, the sleeve 90 is retracted releasing the inflatable member 202. A guidewire, threaded through the injection lumen 209, may be used to ensure that the released inflatable member 202 is properly positioned before the inflatable member 202 is inflated.

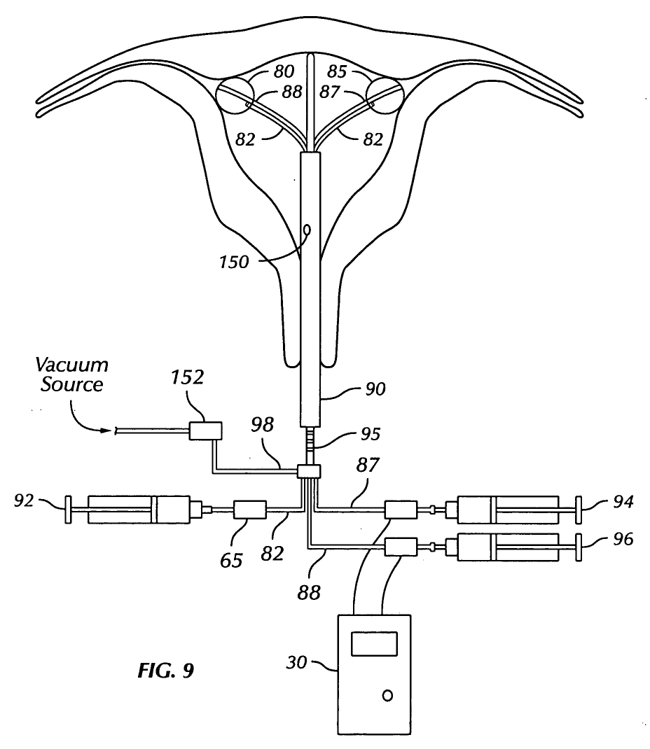

The inflatable member 202 of the patency assessment device 200 may have a number of different configurations. A preferred embodiment of the inflatable member 202 is tear shaped as shown in FIG. 1A. As the inflatable member is inflated the distal end of the inflatable member 202 takes on a conical shape with the fluid injection lumen 209 passing through it. As the inflatable member 202 continues to be inflated the inflation medium pushes radially about the distal end, pushing the distal end into the tubal ostium and pressing against the walls of the fallopian tube as seen in FIG. 10B. The inflatable member 202 is designed such that once it is inflated, its distal end will seal the fallopian tube 135 from the rest of the uterine cavity 130.

The proximal end of the inflatable member 202, or its base, provides a counter balancing force to assist in maintaining the placement of the distal end of the inflatable member 202 inserted into the fallopian tube. As the inflatable member 202 is inflated the base of the inflatable member 202 will bias against the lower portion of the uterus pushing the inflatable member upward.

The inflatable member may be made from compliant or non-complaint materials that would include but not be limited to materials such as latex rubber, silastic rubber, elastomeric rubber, polyurethane, silicone, natural or synthetic rubber, polyester. The surface of the inflatable member 202 could be either smooth or textured. A textured surface would make the positioning of the device more reliable.

An inflation tube 204 is used to carry fluid (i.e., liquid or gas) for inflation of the inflatable member 202. The inflation tube 204 passes from the inflation fluid supply 206, through the housing element 15 and into the inflatable member 202

The inflatable member 202 can be inflated using either a liquid or a gas. Gases that could be used to inflate the inflatable member 202 would include, but not be limited to, carbon dioxide or room air. Liquids for inflation of the inflatable member 202 include, but are not limited to, include sterile water or oil based solutions, saline, dextrose, and glycerin. The medium used for inflation of the inflatable member 202 may vary in viscosity and density.

The injection lumen 209 is used for the injection of fluid into the sealed fallopian tube. In the embodiment shown in FIG. 1A, the inflatable member fits over the distal segment of the injection lumen 209 and is connected to the injection lumen with an airtight connection on a top side and a bottom side. The distal end of the injection lumen 209 extends to the outer surface of the distal end of the inflation member 202, or even beyond the outer surface of the inflatable member 202. The proximal end of the injection lumen 209 extends throughout the housing element 15 and connects to the injection fluid supply 208.

Injection mediums that could be used in the present invention include, without limitation, sterile water or oil based solutions, saline, dextrose, and glycerin, radio-opaque or ultrasound contrast media, pigmented dyes, and medicated liquids or particles. The injected mediums would preferably be pre-warmed to normal body temperature at around 37° C., or alternatively, within the range of 15-45° C.

The inflatable member 202 may have numerous shapes that will allow the inflatable member 202 to conform to the shape of the uterine cavity and fit snugly into the tubal ostium when inflated. The shape of the inflatable member includes, but is not limited to, being spherical, elliptical, triangular or specially designed to conform to the uterine cavity. In addition, the inflation member may contain regions of varying thickness, solidity, flexibility or elasticity as to distend variably in a radial fashion when inflated.

The inflation and injection fluid supplies 206 and 208 of the present invention may include a reservoir for storing fluid (i.e., either liquids or gases) to be injected into the inflatable member 202 and the injection lumen 209 and a means for dispensing such fluids either using manual or automatic control. In a preferred embodiment, simple disposable syringes 205 and 207 are used to supply fluids to the patency assessment device. If automatic control of injection is desired, a device such as a syringe or peristaltic pump could be used to control the inflation of the inflatable member 202 through the syringe 205 and the dispensing of fluid into the sealed fallopian tube through syringe 207. The automatic fluid supply system would enable precise measurements of fluid volumes and flow rates. Injection syringes 205 and 207 may be filled with various mediums, either liquids or gases, to be dispensed simultaneously or independently.

Optionally, guidewires, cannulas or other devices may be inserted into the fallopian tube via the injection lumen 209. For example, a stainless steel or platinum tipped guidewire could be inserted into the sealed fallopian tube in conjunction with an injected fluid medium to assist in dislodging debris or occlusions in the fallopian tube.

Preferred embodiments of the patency assessment device 202 include a monitoring and recording system 30, as shown in FIG. 1A. One embodiment of a monitoring and recording system 30 consists of simple in-line, liquid or air filled, pressure transducers 65 located at the proximal end of the injection lumen 209. The pressure transducer 65 is designed to measure the pressure in the sealed fallopian tube. An additional transducer 65 may be used to measure the pressure in the inflatable member 202. The monitoring system also includes a means for displaying and recording the pressures and/or volumes measured, as well as fluid flow through the injection lumen 209 and the inflation tube 204.

Once a first fallopian tube has been treated or its patency assessed, the inflatable member 202 can be deflated, rotated 180 degrees and re-inflated to treat or test the contralateral fallopian tube.

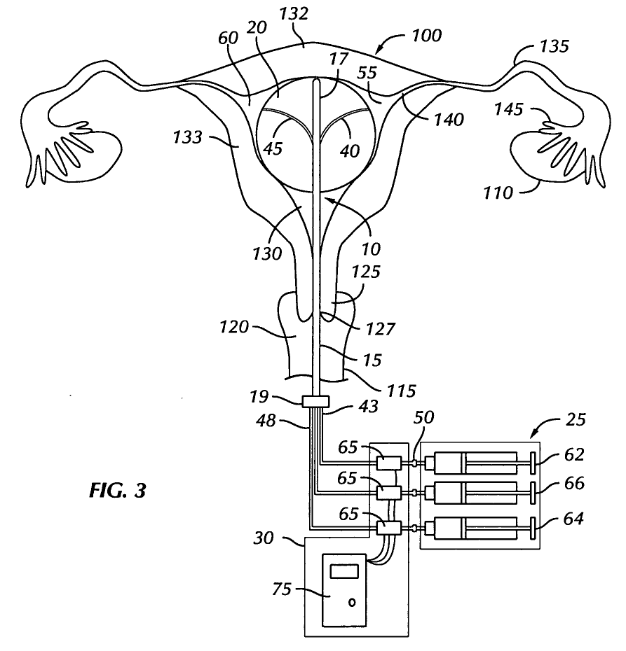

A second embodiment of the present invention that selects and seals one fallopian tube at a time is shown in FIG. 2. The patency assessment device 220 is shown in FIG. 2 vaginally inserted into and extending through the cervical canal and into the uterine cavity 130. Among the major components of the patency assessment device 220 is an elongated flexible housing element 15, a retractable sleeve 90, a cervical plug 222, a cervical plug inflation tube 232, a cervical inflation fluid supply 223 in fluid communication with the cervical plug 222 through the inflation tube 232, a fallopian plug 224, a fallopian plug inflation tube 234, a fallopian inflation fluid supply 225 in fluid communication with the fallopian plug 224 through the inflation tube 234, an injection fluid supply 229, an injection lumen 226, and a pressure monitoring system 30 located between the housing element 15 and the injection fluid supply 229.

The housing element 15 is used to maneuver the patency assessment device 220 through the cervix 125 to the uterine cavity 130. A retractable sleeve 90 is positioned over the distal end of the housing element 15 such that during insertion the retractable sleeve 90 covers the cervical and fallopian plugs 222 and 224 and the injection lumen 226 and the inflation tube 234. Markings 95 on the housing element 15 assist in the proper orientation of the housing element 15. Once the patency device is in place, the sleeve 90 is retracted releasing the injection lumen 226 and the cervical and fallopian plugs 222 and 224.

When the cervical plug 222 is released by the retraction of sleeve 90, it is expanded to fill the base of the uterus and seal the cervical canal. The inflatable member 222 may have a number of different configurations. A preferred embodiment of the cervical plug 222 is an inflatable balloon having substantially a conical shape. As the cervical plug is inflated the narrow pointed end of the cervical plug 222 fills the top end of the cervical canal and is biased against the uterine walls. The cervical plug 222 is designed such that once it is inflated; its proximal end will seal the uterine cavity 130 from the cervix 125.

The fallopian plug 224 may also be conical in shape and is designed to seal the fallopian tube when it is expanded or inflated as shown in FIG. 2. One embodiment of the fallopian plug is an inflatable balloon situated at the distal end of an inflation tube 234. The inflation tube 234 is preferably made of a shape memory metal such as nitinol that is strong enough to assist in the proper positioning of the fallopian plug 224.

The cervical plug 222 and the fallopian plug 224 may be made from materials that would include, but not be limited to, latex rubber, silastic rubber, elastomeric rubber, polyurethane, silicone, natural or synthetic rubber, or polyester. The surface of the cervical plug 222 and fallopian plug 224 can be either smooth or textured. The cervical and fallopian plugs may contain regions of varying thickness, solidity, flexibility or elasticity as to distend variably in a radial fashion when inflated.

The cervical plug 222 and the fallopian plug 224 can be inflated using either a liquid or a gas. Gases that could be used to inflate the cervical and fallopian plugs would include, but not be limited to, carbon dioxide or room air. Liquids for inflation of the cervical and fallopian plugs include, but are not limited to, sterile water or oil based solutions, saline, dextrose, and glycerin. The medium used for inflation of the cervical and fallopian plugs may be the same medium or different mediums. Likewise, each inflation medium may vary in viscosity and density.

The inflation tubes 232 and 234 are used to carry fluid (i.e., liquid or gas) for inflation of the cervical plug 222 and the fallopian plug 224 respectively. In the embodiment shown in FIG. 2, the inflation tube 232 passes from the inflation fluid supply 223, through the housing element 15 and into the cervical plug 222. Similarly, the inflation tube 234 passes from the inflation fluid supply 225, through the housing unit, and extends upward towards the tubal ostium and into the fallopian plug.

The injection lumen 226 extends through the cervical plug 222 and into the uterine cavity 130. The injection lumen 226 is used for the injection of fluid into the uterine cavity and into the unsealed fallopian tube. The proximal end of the injection lumen 226 extends through the housing element 15 and connects to the injection fluid supply 229.

The inflation and injection fluid supplies of the present invention includes a reservoir for storing fluid (i.e., either liquids or gases) to be injected into the cervical and fallopian plugs 222 and 224 and the injection lumen 226, as well as a means for dispensing such fluids either using manual or automatic control. In a preferred embodiment, simple disposable syringes 221, 227 and 228 are used to supply fluids to the patency assessment device. If automatic control of injection is desired, a device such as a syringe or peristaltic pump could be used to control the dispensing of fluid into the cervical and fallopian plugs or into the uterine cavity and fallopian tube. An automatic fluid supply system would enable precise measurements of fluid volumes and flow rates. Preferred embodiments of the patency assessment device 220 include a monitoring and recording system 30, as shown in FIG. 2.

Once a first fallopian tube has been treated or its patency assessed, the fallopian plug can be deflated or otherwise disengaged, rotated 180 degrees and re-inflated or repositioned to treat or test the contralateral fallopian tube.

B. Devices Sealing Both Fallopian Tubes

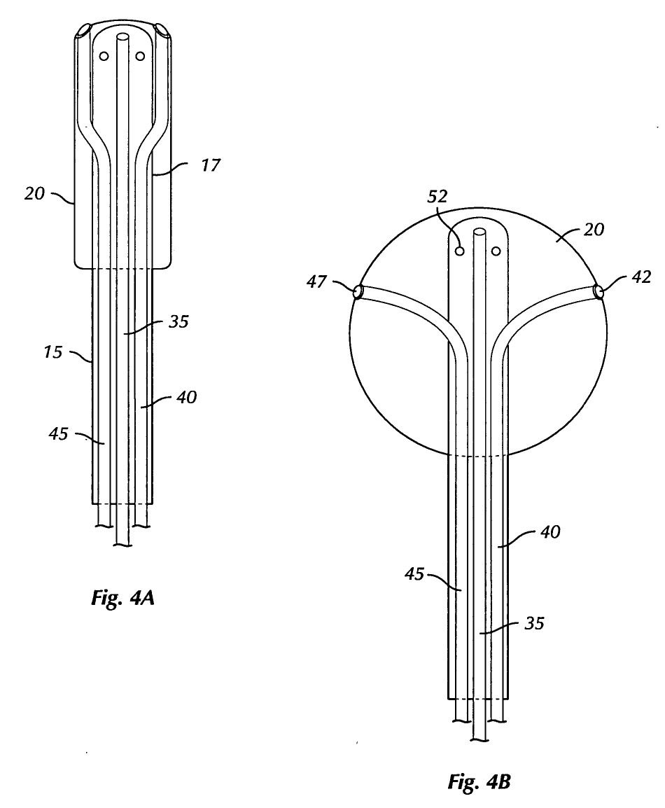

A first embodiment of the present invention that selects and seals both fallopian tubes at the same time is shown in FIG. 3. The patency assessment device 10 of the present invention is vaginally inserted and extends through the cervix 125 into the uterine cavity 130. Among the major components of the patency assessment device 10 is an elongated flexible housing element 15, an inflatable member 20 located on a distal segment 17 of the housing element 15, a fluid supply 25 connected to the proximal end 19 of the housing element 15, and a pressure monitoring system 30 located between the housing element 15 and fluid supply 25.

The housing element 15 is used to maneuver the inflatable member 20 through the cervix 125 and into the uterine cavity 130. The housing element 15 may be made of any rigid, semi-rigid, or flexible material, such as polyurethane, polyethylene, polyimide, or other polymers, plastics or metals. The housing element may be either internally and/or externally coated with specialized friction reducing hydrophilic or hydrophobic materials. Examples of such materials are Teflon from Dupont Chemical Company or Lubrilast from AST Products, Inc. The distal tip of the housing element 15 is preferably shaped with a rounded, non-traumatic end to protect the fundus 132 whenever the distal tip contacts and abuts the fundus.

The length of the housing element 15 must be adequate to extend through the patient's vagina 115 and cervix 125 to reach the uterine cavity 130, as well have a sufficient length to conveniently connect to the fluid supply 25. The length of the patency assessment device to be used on humans would typically have a housing element 15 ranging from about 15 cm to about 30 cm in length.

The size of the outside diameter of the housing element 15 requires one to consider the general diameter of an instrument that can be inserted through the cervical canal in a comfortable and non-traumatic fashion. The patency assessment device for use in humans would generally have an outside diameter that ranges from 2 mm to about 1 cm, and would preferably be approximately 4 mm.

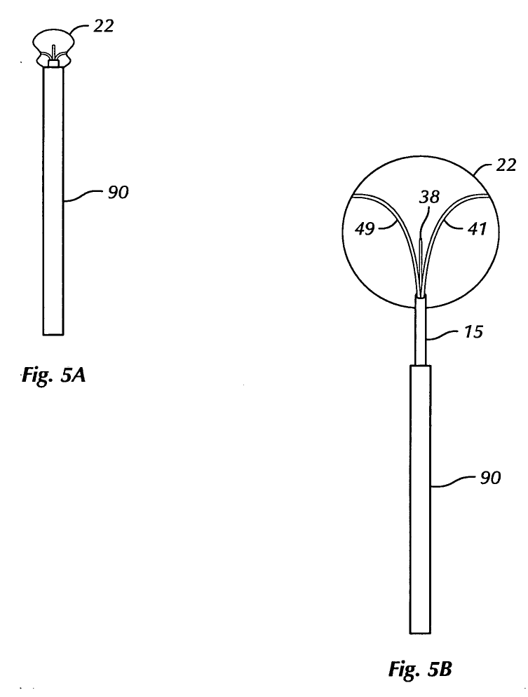

Referring to FIGS. 4A and 4B, a preferred embodiment of the patency assessment device 10 has three internal tubular structures. A balloon inflation tube 35 is used to carry fluid (i.e., liquid or gas) for inflation of the inflatable member 20 located on the distal segment 17 of the housing element 15. In the embodiment shown in FIGS. 4A and 4B, the inflatable member fits over the distal segment 17 and is connected to the housing element 15 with an airtight connection. The distal segment 17 has a communication means from the inflation tube 35 to the interior of the inflatable member 20. This communication means, such as pores 52, provide the means for fluid to enter the interior of the inflatable member 20 to distend the inflatable member to conform to the shape of the uterine cavity.

A right injection lumen 40 and left injection lumen 45 are used for the selective injection of fluid into either the right or left fallopian tube. The distal end 42 of the right injection lumen 40 and the distal end 47 of the left injection lumen 45 extend outside of the housing element 15 to terminate and open out onto the outer surface of the inflatable member 20. The proximal end 43 of the right injection lumen 40 and the proximal end 48 of the left injection lumen 45 terminate in connections 50 for the fluid supply 25. The inflation tube 35, right injection lumen 40, and left injection lumen 45 extend throughout the housing element 15.

Referring back to FIG. 3, the inflatable member 20 of the patency assessment device is designed such that once it is inflated, two non-communicating regions, a right cornual region 55 and a left cornual region 60, of the uterine cavity are created. The inflatable member may be made from compliant or non-complaint materials that would include but not be limited to materials such as latex rubber, silastic rubber, elastomeric rubber, polyurethane, silicone, natural or synthetic rubber, polyester. The surface of the inflatable member 20 could be either smooth or textured. A textured surface would make the positioning of the device more reliable.

The inflatable member 20 can be inflated using either a liquid or a gas. Gases that could be used in the present invention would include, but not be limited to, carbon dioxide or room air. Liquids for inflation of the inflatable member 20 that could be used in the present invention would include, but not be limited to, sterile water or oil based solutions, saline, dextrose, and glycerin. The medium used for inflation of the inflatable member 20 may vary in viscosity and density.

Injection mediums that could be used in the present invention include, without limitation, sterile water or oil based solutions, saline, dextrose, and glycerin, radio-opaque or ultrasound contrast media, pigmented dyes, and medicated liquids or particles. The injected mediums would preferably be pre-warmed to normal body temperature at around 37° C., or alternatively, within the range of 15-45° C.

The inflatable member 20 may have numerous shapes that will allow the inflatable member 20 to conform to the shape of the uterine cavity when inflated. The shape of the inflatable member includes but is not limited to being spherical, elliptical, triangular or specially designed to conform to the uterine cavity. In addition, the inflation member may contain regions of varying thickness, solidity, flexibility or elasticity as to distend variably in a radial fashion when inflated.

The fluid supply 25 of the present invention may include a reservoir for storing fluid (i.e., either liquids or gases) to be injected into the inflatable member and the injection lumens and a means for dispensing such fluids either using manual or automatic control. In a preferred embodiment, simple disposable syringes 62, 64 and 66 are used to supply fluids to the patency assessment device. If automatic control of injection is desired, a device such as a syringe or peristaltic pump could be used to control the inflation of the inflatable member 20 through the syringe 66 and the dispensing of fluid into the two non-communicating regions 55 and 60 through syringes 62 and 64 respectively. The automatic fluid supply system would enable precise measurements of fluid volumes and flow rates. Injection syringes 62 and 64 may be filled with various mediums, either liquids or gases, to be dispensed simultaneously or independently into the cornual regions 55 and 60.

Optionally, guidewires, cannulas or other devices may be inserted into one of the fallopian tubes via the same working channels and lumens as the gas or liquid mediums. For example, a stainless steel or platinum tipped guidewire could be inserted into a fallopian tube in conjunction with an injected fluid medium to assist in dislodging debris or occlusions in the fallopian tube.

Preferred embodiments of the patency assessment device 10 include a monitoring and recording system 30, as shown in FIG. 3. One embodiment of a monitoring and recording system 30 consists of simple in-line, liquid or air filled, pressure transducers 65 located at the proximal end of the inflation tube and injection lumens. The pressure transducers 65 are designed to measure the pressure in the inflatable member 20, right cornual region 55, and left cornual region 60. The monitoring system also includes a means for displaying and recording 75 the pressures and/or volumes measured in all three regions and the flows through the two injection lumens and the inflation tube.

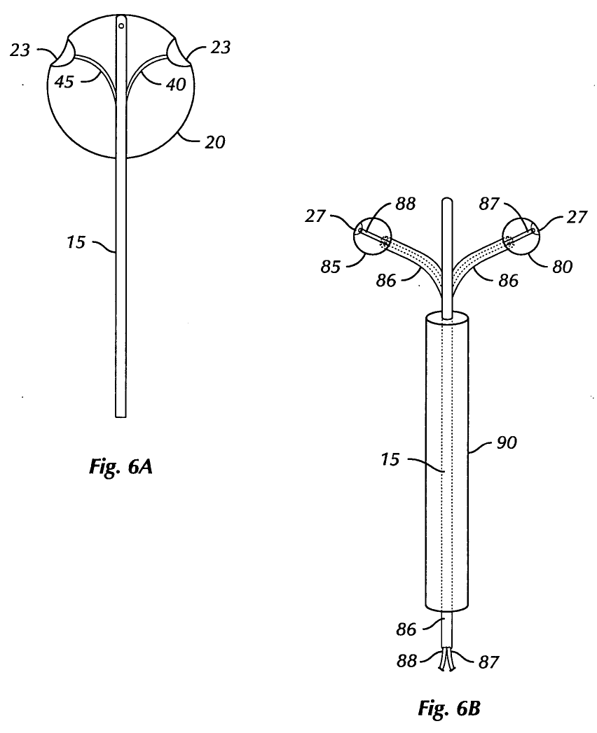

FIG. 5A illustrates an alternative embodiment of the patency assessment device 10 where a deflated inflatable member 22 is positioned at the distal tip of the housing element 15. A retractable sleeve 90 may be positioned over the housing element 15, as well as a part or the entire deflated inflatable member 22. Once the patency device is in place, the retractable sleeve 90 is retracted and the inflatable member 22 is inflated as shown in FIG. 5B. The positioning of the inflatable member 22 at the distal tip of the housing element 15 allows the inflatable member 22 to be more easily positioned and moved to the correct location within the uterine cavity 130.

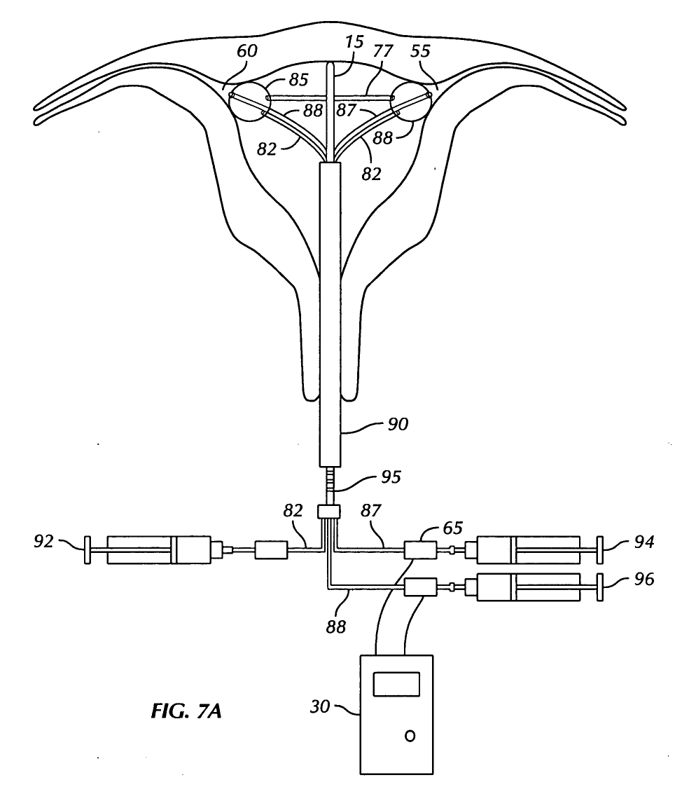

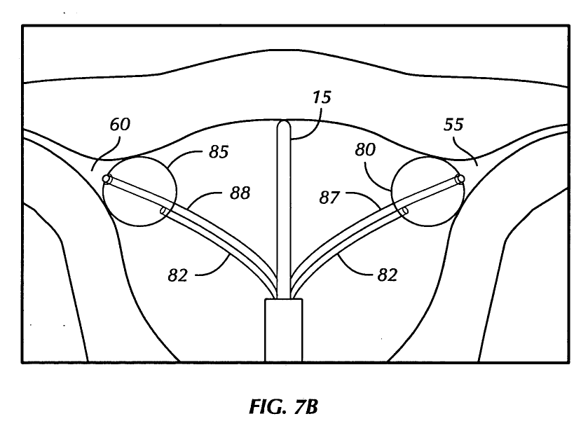

The construction of the inflatable member 22 and its constituent right injection lumen 41 and left injection lumen 49 is similar in many aspects to that described for inflatable member 20 and injection lumens 40 and 45 of FIG. 3, yet it differs in its inflation means. The inflatable member 20 is inflated by fluid, either liquid or gas, passing through the housing element 15 through the pores 52 and into the inflatable member 20; whereas the inflatable member 22 is inflated by fluid passing through an inflation tube 38 into the inflatable member 22. The inflation tube may be an independent tubular structure as shown in FIG. 5B or it may be a part of a tubing structure concentric about the injection lumen such as illustrated in FIG. 6B for another embodiment of the invention.

FIG. 6A illustrates an alternative embodiment of the inflatable member 20 of the present invention. The inflatable member 20 is formed to include cavities 23 at the location of the exit sites of the injection lumens 40 and 45. The cavities are concave regions on the outer surface of the inflatable member that ensure a sufficient open region between the inflatable member 20 and the uterine wall for free fluid flow.

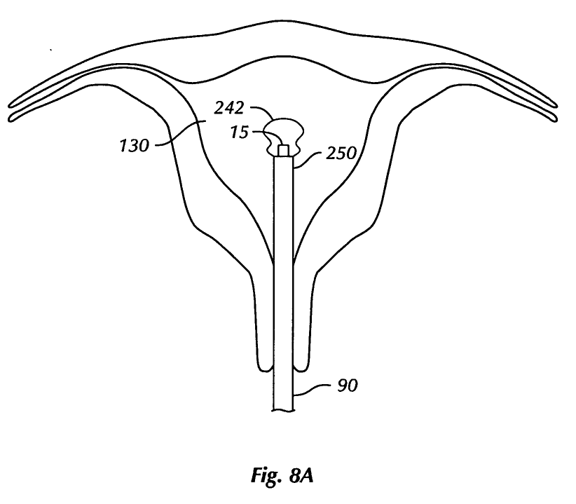

A second embodiment of the present invention that selects and seals both fallopian tubes at the same time is shown in FIGS. 7A, 7B, and 6B. FIGS. 7A and 7B illustrate an embodiment of the present invention in which an independent right inflatable member 80 and an independent left inflatable member 85 are used to create the two non-communicating cornual regions 55 and 60. The two inflatable members are respectively mounted on a right injection lumen 87 and left injection lumen 88. A retractable sleeve 90 is positioned over the housing element 15 such that during insertion the retractable sleeve 90 covers the two inflatable members 80 and 85 and the two injection lumens 87 and 88. The housing element 15 is preferably longer than the sleeve 90 such that the distal tip of the housing member 15 will contact the upper fundus 132 when the patency device is in place. Markings 95 on the housing element 15 assist in the proper orientation of the housing element 15. Once the patency device is in place, the sleeve 90 is retracted releasing one injection lumen and one inflatable member on each side of the housing element 15 into the desired regions.

An inflation tube 82 is used to carry fluid for inflation of the right and left inflation members 80 and 85. The inflation tube 82 is in communication with a fluid supply, such as the syringe 92. The inflation tube 82 is a single tube extending through the housing element 15 until the inflation tube 82 approaches the distal segment of the housing element 15 where the inflation tube is bifurcated and one end extends out of the right side of the housing element and one end extends out of the left side of the housing element 15. The right distal end of the inflation tube 82 runs alongside the right injection lumen 87 from the distal segment of the housing element into the inflatable member 80. The right side of the distal end of the inflation tube 82 enters the right inflation member 80 and provides fluid communication from the syringe 92 to the right inflation member 80. Similarly, the left side of the distal end of the inflation tube 82 enters the left inflation member 85 and provides fluid communication from the syringe 92 to the left inflation member 85.

Alternatively, the embodiment of the present invention shown in FIG. 7A may have an independent right inflation tube for the inflation of the inflatable member 80 and an independent left inflation tube for the inflation of the inflatable member 85. If the inflatable members 80 and 85 have independent inflation tubes the right and left inflatable members 80 and 85 may be inflated separately or concurrently. In addition, the two inflatable members may be inflated at different pressure levels.

The device shown in FIG. 7A differs from the device shown in FIG. 7B by having bias line 77 extending from the housing element 15 on the right side to the inflatable member 80 and on the left side to the inflatable member 85. Bias line 77 is a tubular structure that may have an open end that penetrates the inflatable member and a closed end attached to the housing element 15 as shown in FIG. 7A. This construction allows the line 77 to fill with fluid as the inflatable member is filled. As bias line 77 is filled, it will bias the inflatable member away from the housing element 15 and towards the tubal ostium. Although not illustrated in the figures, bias line 77 may alternatively be closed at the end attached to each of the inflatable members and may have another end that opens into the tubular housing element such that bias line 77 is filled with fluid passing through the housing element. As bias line 77 fills with fluid, bias line 77 is pushed away from the housing element and thereby biases the inflatable members 80 and 85 outward towards opposed tubal ostiums.

The injection lumen 87 is connected on a proximal end to a pressure transducer 65 and a fluid supply such as syringe 94. The injection lumen 87 travels through the housing element 15 until the injection lumen 87 approaches the distal segment of the housing element 15 where the injection lumen 87 exits the housing element 15. The inflatable member 80 fits over the distal end of the injection lumen 87. There is an airtight connection between both the proximal and the distal surfaces of the inflatable member 80 and the external surface of the injection lumen 87. The injection lumen 87 extends from the housing element 15 through the inflatable member 80. The distal end of the injection lumen 87 terminates onto the outer surface of the inflatable member 80 to open out into the right cornual region 55 when the patency device 10 is in place.

The injection lumen 88 is connected on a proximal end to a pressure transducer 65 and a fluid supply such as syringe 96. The injection lumen 88 travels through the housing element 15 until the injection lumen 88 approaches the distal segment of the housing element 15 where the injection lumen 88 exits the housing element 15 on the opposite side from where the injection lumen 87 exits the housing element 15. The inflatable member 85 fits over the distal end of the injection lumen 88 with an airtight connection. The injection lumen 88 extends from the housing element 15 through the inflatable member 85. The distal end of the injection lumen 88 terminates onto the outer surface of the inflatable member 85 to open out into the right cornual region 60 when the patency device 10 is in place.

Similarly, FIG. 6B illustrates an alternative embodiment of the inflatable members 80 and 85 of the present invention. The inflatable member 80 and 85 are formed to include cavities 27 at the location of the exit sites of the injection lumens 87 and 88. The cavities 27 are concave regions on the outer surface of the inflatable members that ensure a sufficient open region between the inflatable members 80 and 85 and the uterine wall to ensure a flow path for the contrast media, medications and the like being injected into the cornual regions. Furthermore, FIG. 6B illustrates how the inflation tube can surround the injection lumens 87 and 88 to form a concentric tubular structure having an inflation tube surrounding the injection lumen.

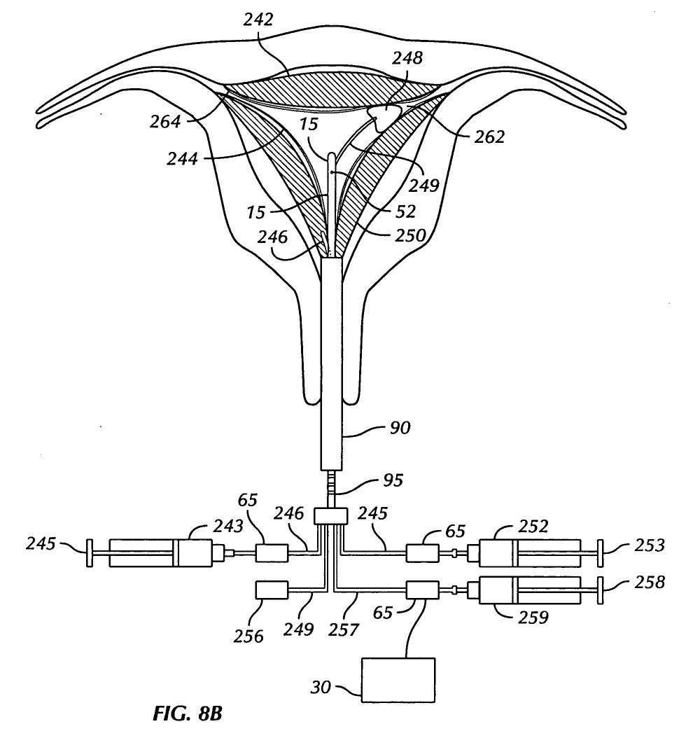

A third embodiment of the present invention that selects and seals both fallopian tubes at the same time is shown in FIGS. 8A, 8B and 8C. Among the major components of the patency assessment device 250 is an elongated flexible housing element 15 having at least one pore 52, a retractable sleeve 90, an inflatable member 242 attached to the exterior of the housing element 15, multiple supporting structures 244, an inflation tube 246, a guide line 249, and an expandable plug 248.

The housing element 15 is used to maneuver the inflatable member 242 through the cervix 125 to the uterine cavity 130. The housing element 15 may be made of any rigid, semi-rigid, or flexible material. The distal tip of the housing element 15 is preferably rounded and non-traumatic to the interior of the inflatable member 242 whenever the distal tip contacts and abuts the inflatable member 242.

A retractable sleeve 90 is preferably positioned over the housing element 15, as well as a part or the entire deflated inflatable member 242. Once the patency device 250 is in place, the retractable sleeve 90 is retracted and the inflatable member 242 is inflated as shown in FIGS. 8A and 8B.

The inflatable member 242 is substantially triangular in shape having two conical regions, one for insertion into each tubal ostium to form a sealed cornual region, and a conical region at the base of the inflatable member 242 to seal the interior of the inflatable member 242 from the cervical canal. As the inflatable member 242 is inflated the radially expanding conical regions ensure a fluid-tight seal around the critical cornual regions.

The inflatable member 242 contains multiple supporting structures 244 that are foldable within the inflatable member 242 during deployment of the patency assessment device 250 and expandable as the inflatable member 242 is unfolded. An example of such supporting structures 244 are the two shape memory metal wires (e.g., nitinol wires) shown in FIG. 8B. The supporting structures 244, or edge wires, have an outward bias that curves away from the midline. The two edge wires can be used to visualize, manipulate and align the inflatable member 242 outside of the body using a hysteroscope. Once the inflatable member 242 is properly aligned such that the conical regions seal the cornual regions, the inflatable member 242 is inflated. The two metal wires support the outer edges of the triangular inflatable member 242 as it is being inflated within the uterine cavity 130.

The construction of the inflatable member 242 and its constituent right injection inlet 262 and left injection inlet 264 is similar in many aspects to that described for inflatable member 20 and injection lumens 40 and 45 of FIG. 3. The inflatable member 242 is inflated by fluid, either liquid or gas, passing through an inflation tube 246 into the inflatable member. The inflation tube 246 is in fluid communication with the inflatable member 242 and an inflation fluid supply 243 controlled by an inflation control means 245. The inflation control means may be either an automatic or a manual control means, such as the syringe 245 illustrated in FIG. 8B.

The inflatable member also contains a guide wire 249 and an expandable plug 248. The guide wire 249 is preferably a shaped memory metal tubular structure having an expandable conical plug 248 at the distal end of the guide wire 249. The manipulation of the guide wire 249 is controlled outside of the body using manual or a remote control guide line manipulator 256. The expandable plug may be mechanically folded and expanded, or it may be inflated and deflated. For example, fluid may be passed through the lumen of the guide wire 249 into the plug 248 to inflate the plug. The lumen of the guide wire 249 is connected to a fluid supply 252 through a fluid dispensing line 254. Inflation of the plug 248 is controlled by either an automatic or a manual means, such as the syringe 253.

Once the patency assessment device 250 is deployed and the expandable plug is in place and has sealed off either the right injection inlet 262 or the left injection inlet 264, fluid (i.e., liquid or gas) is injected into the interior of the inflatable member 242 through a communication means from an injection fluid supply 259 to the interior of the inflatable member 250. This communication means (e.g., an injection line transversing the housing element and communicating with the interior of the inflatable member 242 through pores 52 in the distal portion of the housing unit 15) provides the means for fluid to enter the interior of the inflatable member 242 and on up into the open injection inlet into the unsealed fallopian tube.

The fluid supply 259 may include a reservoir for storing fluid (i.e., either liquids or gases) to be injected into the interior of inflatable member 242 and the injection inlets and a means for dispensing such fluids either using manual or automatic control. In a preferred embodiment, a simple disposable syringe 258 is used to supply fluids to the interior of inflatable member 242. If automatic control of injection is desired, a device such as a syringe or peristaltic pump could be used to control the injection of fluid into the interior of the inflatable member 242 and through the open injection inlet to be dispensed into the unsealed fallopian tube. Thus, the open fallopian tube may be treated with medicated fluids or the patency of the open fallopian tube may be assessed. The automatic fluid supply system would enable precise measurements of fluid volumes and flow rates into the open fallopian tube.

Preferred embodiments of the patency assessment device 250 include a monitoring and recording system 30, as shown in FIG. 8B. One embodiment of a monitoring and recording system 30 consists of simple in-line, liquid or air filled, pressure transducers 65 located at the proximal end of the injection lumen 257, the guide wire 249 and the inflation tube 246. The pressure transducers 65 are designed to measure the pressure inside the inflatable member 242, the plug 248 and the open or unsealed fallopian tube. The monitoring system also includes a means for displaying and recording the pressures and/or volumes measured.

) Once one fallopian tube has been treated or its patency determined, the expandable plug 248 is closed and the guide line 249 is used to retract the plug 248, rotate it about 180 degrees and redeploy the plug 248 into the contralateral injection inlet of the inflatable member 242 to test or treat the unsealed contralateral fallopian tube. Whenever the treatment or testing is completed, the patency assessment device 250 is deflated and refolded within the retractable sleeve 90 and the device 250 is removed from the patient's body.

C. Ensuring a Fluid-Tight Sealing of the Fallopian Tube

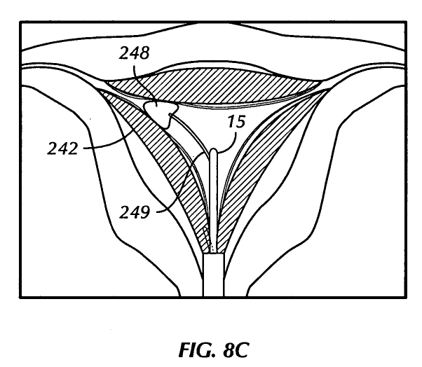

FIG. 9 illustrates an embodiment of the patency device 10 that can be used to test for fluid leakage during pressure testing of the cornual region sealed by the patency assessment device. The patent assessment device of FIG. 9 is similar to the patency device shown in FIG. 7B, with the addition of at least one suction port 150 in the outside surface of the housing element 15. The suction ports 150 are in fluid communication with a vacuum source through vacuum tubing 98 as illustrated in FIG. 9. The suction port 150 is used in determining if a good seal is made between the inflatable member(s) of the patency assessment device and the cornual regions. The suction port 150 is provided on the housing element 15 at a location that is proximal to the inflatable member(s) and just distal to the end of the cervical canal when the patency device 10 is in place. During assessment of patency, a colored liquid is injected through the injection lumen. If a good seal is not present, then colored liquid will leak into the uterine cavity and will be sucked into the suction port and through the vacuum tubing 98 to a clear viewport 152, or other fluid trap, located outside of the patient where the operator can see the colored liquid. Alternatively, if gas is used as the patency testing fluid, a soap agent could be placed in the uterus and the uterus sealed from the cervical canal, such that if the gas leaked the leaking gas would cause air bubbles to form in the soap agent.

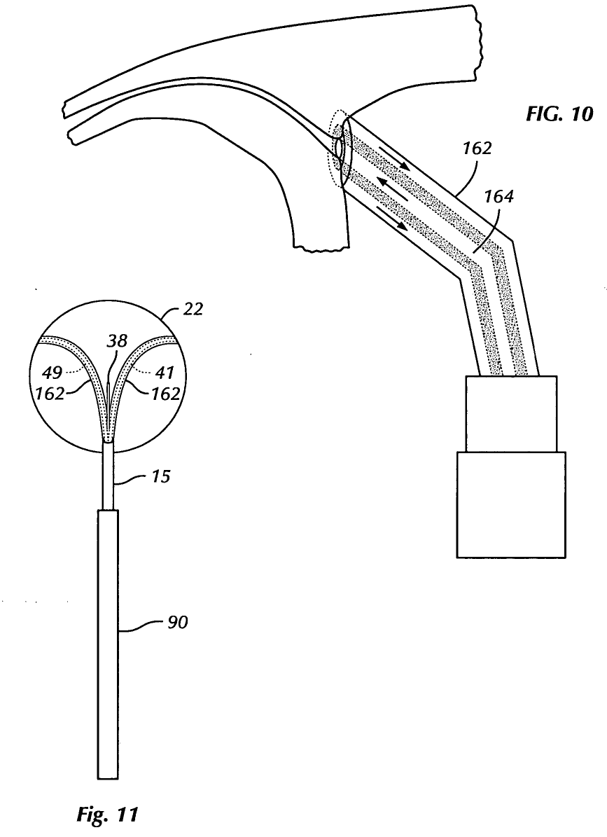

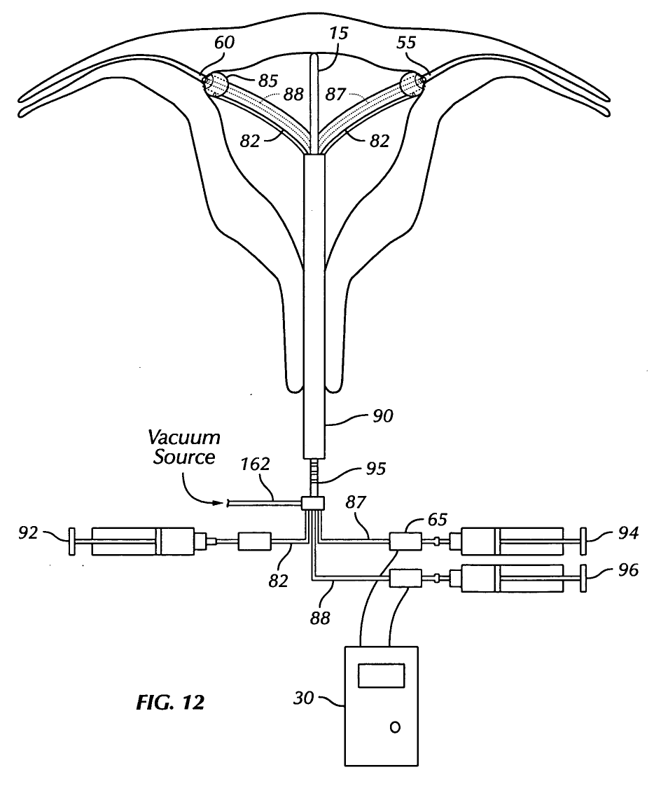

FIG. 10 illustrates another aspect of the present invention. The sealing mechanism shown in FIG. 10 facilitates forming a good seal at the tubal ostium. The patency assessment device of FIG. 10 comprises a tubing structure having a vacuum line 162 concentric with the injection lumen 164. The inner lumen 164 is used to deliver fluid for accessing patency or treating the fallopian tube. The outer vacuum line 162 is designed to engage tissue surrounding the tubal ostium and can be independently connected to a vacuum or suction creating a fluid tight seal. The single device shown in FIG. 10 could be used for both fallopian tubes by rotating the device after use. Alternatively the device could include two similar but separate tubing structures for concurrent use in assessing the patency of both fallopian tubes. Furthermore, the concentric tubular structures, one for vacuum sealing the inflatable member against the tissue surrounding the tubal ostium and the other for injecting fluid (i.e., a gas or liquid) or other material into the fallopian tube, can also be used in inflatable member(s) 20, 22, or 80 and 85 as exemplified in FIGS. 11 and 12.

D. Operation of the Treatment or Patency Assessment Device

In order to operate the device of the present invention, the housing element 15 carrying the inflatable member(s) 20, 22, 80 and 85, 200, 202, 220, 222 or 242 is initially inserted through the vagina 115 and cervix 125 into the uterine cavity 130. The housing element 15 is inserted either blindly or under visual control using methods that would include, but not be limited to, a hysteroscope, X-ray system, or ultrasound. The use of such visual control systems is within the ordinary skill of one who would use this device and is not shown specifically in the figures of the present invention. The housing element 15 is rotated using markings 95 located on the housing element 15 such that the inflatable member(s) are in the correct orientation with respect to the anatomy of the uterine cavity 130.

The inflatable member(s) 20, 22, 80 and 85, 200, 202, 220, 222 or 242 are inflated when the distal end of the housing element 15 is properly positioned within the uterine cavity 130. Inflation of inflatable member(s) 20, 22, 80 and 85, 202, or 242 result in the formation of one or two separate non-communicating cornual regions within the uterus, a right cornual region 55 and/or a left cornual region 60 within the uterine cavity 130. The pressure applied to the inflatable member in order to produce inflation can range from 0 to about 350 mmHg but will preferably range from about 200-250 mm Hg.

Verification of fallopian tube regional isolation and non-communication can be accomplished using a combination of colored and uncolored mediums. For example, a particular fallopian tube 135 that is open to the sealed cornual region 55 could be injected with a blue dye and monitored for any leakage of the blue dye using the suction port 150 and the viewport 152. Likewise, the other sealed cornual region 60 can be injected with a clear or differently colored liquid and the suction port 150 monitored for leaking clear or differently colored liquid.

Alternatively, proper isolation of each tubal region could be performed using a low pressure test where the right and left cornual regions are brought up to a low pressure that would not open the fallopian tubes (20-40 mm Hg). The pressure is then monitored and changes in pressure would reveal the improper sealing of the region in which the pressure changed.

In order to evaluate the patency of each fallopian tube, fluid is injected through the injection lumen towards the right cornual region or left cornual region. The pressure in the region is increased slowly by continuing to inject fluid into the region over a period of 1-2 minutes. The pressure is increased to a maximum of 200 mm Hg and held at this level for an additional 3-5 minutes. If the pressure does not drop then the tube is considered to be closed or occluded. If the tube opens, then the pressure will drop to just below the minimum threshold pressure for opening the tube. The final pressure maintained in the cornual region will indicate the degree of patency of the fallopian tube. The process is then repeated in the contralateral fallopian tube thereby giving the physician not only an indication of whether the tubes are open but also which tube and to what degree.

It has been observed that if fallopian tubes are patent, carbon dioxide may flow through the tubes and enter the peritoneal cavity. The release of gas may produce some pain in one or both shoulders of the patient. This test, known as the Rubin test, could be used with the device of the present invention. If the manometer used with the Rubin Test registers less than 100 mm Hg, the tubes are patent. But, if the test shows the pressure in the fallopian tubes to be between 120 and 130 mm Hg there may be stenosis or stricture, but not complete occlusion. If the test pressure rises to 200 mm Hg, the fallopian tubes are determine to be completely occluded (Rubin, I. C. 1983. Non-operative determination of patency of fallopian tubes in sterility: intrauterine inflation with oxygen and production of an artificial pneumoperitoneum. JAMA 250(17): 2358). Thus, based on the data of Rubin, if a fallopian tube is only partially closed due to a condition such as inflammation; the pressures monitored by the device of the present invention would be expected to be in the range of 120 to 130 mm Hg on a manometer.

The device of the present invention has advantages over devices currently in use in that it isolates and independently evaluates patency in each fallopian tube without the need for x-rays or contrast media. All previous devices for use in hysterosalpingography inject a contrast fluid into the entire uterus and use X-rays to visualize the flow. However, one of skill would understand that it is important to know the condition of each fallopian tube independent of the other. By injection of contrast media into the uterus, previous approaches to evaluate fallopian tube patency in both tubes at the same time and evaluation of flow through a given tube can only be confirmed using X-ray visualization. Also since X-rays must be used sparingly, there is a chance that the tubes may look closed during the injection and visualization due to tubal spasm. The device of the present invention also has the advantage of being capable of determining fallopian tube patency by monitoring changes in pressure, rather than visualization only through use of X-rays. As a result, there are no limitations on use of this device in terms of the time needed for a procedure, thus enabling a physician to rule out closure due to tubal spasm since the monitoring of pressure can occur over a longer period of continuous time rather than only at one point in time.

Additionally, the device of the present invention will give quantitative measures of the degree of patency of the fallopian tubes. By measuring pressure and flow rates one can obtain more information on the physiologic state of the tube than simple visualization used in hysterosalpingography. Knowing the exact pressure at which the tube opens gives an indication of the degree of closure of the tube.

Although the present invention and its advantages have been described in detail, it should be understood that various changes, substitutions and alterations can be made herein without departing from the spirit and scope of the invention as defined by the appended claims.

1. An apparatus for selectably accessing a fallopian tube in a patient, said device comprising:
a) a flexible housing element having a top end and a bottom end;
b) an inflatable member having a distal end and a proximal end, wherein the distal end has a conical tip and the proximal end has an enlarged base such that whenever the inflatable member is inflated in a uterus the conical tip is biased towards a tubal ostium of a first side of the uterus and the enlarged base is biased against an opposed side of the uterus, whereby a cornual region is sealed from the uterine cavity;
c) an injection lumen passing through the housing element and the inflatable member to terminate on an outer surface of the distal end of the inflatable member and open into the sealed cornual region;
d) an inflation line in fluid communication between an inflation fluid supply and the inflatable member; and
f) an injection mechanism for controllably injecting a fluid through the injection lumen and into the sealed cornual region.

2. The apparatus of claim 1, further comprising a retractable sleeve reciprocable between a first and second position, wherein the first position is located over a distal portion of the housing element and covers a portion of the deflated inflatable member and the second position is proximal to the first position and uncovers the deflated inflatable member.

3. An apparatus for selectably accessing a fallopian tube in a patient, said device comprising:
a) a flexible housing element having a top end and a bottom end;
b) an inflatable member having a distal end and a proximal end, wherein the inflated inflatable member has a first conical tip on the distal end, the conical tip biased into a first tubal ostium to seal a cornual region;
c) a first injection lumen passing through the housing element and the inflatable member to terminate on an outer surface of the distal end of the inflatable member at the first conical tip and open into the sealed cornual region;
d) an inflation line for inflating the inflatable member; and
f) an injection means for controllably injecting a fluid through the injection lumen and into the sealed cornual region.

4. The apparatus of claim 3, wherein the inflated inflatable member further comprising a second conical tip biased into a second tubal ostium and a second injection lumen passing through the inflatable member and terminating on the outer surface of the distal end of the inflatable member at the second conical tip.

5. An apparatus for selectably accessing a fallopian tube in a patient, said device comprising:
a) an expandable cervical plug that serves to seal a uterine cavity from a cervical canal whenever expanded;
b) an expandable tube plug that serves to seal an entrance to a tubal ostium from the uterine cavity whenever expanded;
c) a guide line attached to the tube plug for maneuvering the plug into the entrance to the tubal ostium;
d) a tubular elongated flexible housing element having a distal segment that transverses the cervical plug, wherein the housing element is in fluid communication with an injection fluid supply and the uterine cavity;
d) a retractable sleeve reciprocable between a first and second position, wherein the first position is located over a distal portion of the housing element and covers the cervical plug and the tube plug and the second position is proximal to the first position and uncovers the cervical plug or the tube plug; and
e) an injection means for injecting a fluid through the tubular housing element and into the uterine cavity.

6. The apparatus of claim 5, wherein the expandable cervical plug is a balloon.

7. The apparatus of claim 5, wherein the expandable tube plug is a balloon.

8. The apparatus of claim 7, wherein the guide line is tubular and in fluid communication between an inflation fluid supply and the tube plug.

9. The apparatus of claim 5, wherein the guide line is made of a shape memory metal.

10. An apparatus for selectably accessing a fallopian tube in a patient, said device comprising:
a) a flexible housing element having a distal end and a proximal end;
b) an inflatable member disposed on a distal segment of the housing element;
c) an inflation means for inflating the inflatable member;
d) a first injection lumen passing through the inflatable member and terminating on an outer surface of the inflatable member;
e) a second injection lumen passing through the inflatable member and terminating on the outer surface of the inflatable member separated from the first injection lumen; and
f) an injection means for selectably injecting a fluid into the first or the second injection lumen.

11. The apparatus of claim 10, wherein the inflation means is a tubular line providing fluid communication between a fluid supply and the inflatable member.

12. The apparatus of claim 10, wherein the first and second injection lumens terminate on opposite sides of the inflatable member whenever the inflatable member in inflated.

13. The apparatus of claim 10, further comprising a retractable sleeve reciprocable between a first and second position, wherein the first position is located over a distal portion of the housing element and covers a portion of the deflated inflatable member and the second position is proximal to the first position and uncovers the inflatable member.

14. The apparatus of claim 10, wherein the inflatable member has two concave regions on the outer surface of the inflatable member, the first injection lumen terminating in one concave region and the second injection lumen terminating in the second concave region.

15. The apparatus of claim 10, further comprising a vacuum line traveling from the distal outer surface of the inflatable member through the inflatable member and the housing element to a vacuum source proximal to the housing element.

16. The apparatus of claim 15, wherein the vacuum line surrounds the first and second injection lumens.

17. A method of assessing patency of fallopian tubes in a patient comprising:
a) inserting a distal portion of the apparatus of claim 10 into a uterine cavity of the patient;
b) orienting the apparatus with respect to the uterus anatomy;
c) aligning the first injection lumen with a first tubal ostium of a first fallopian tube;
d) inflating the inflatable member to seal the first fallopian tube from the uterine cavity;
e) injecting a first fluid medium through the first injection lumen of the inflatable member until a first predetermined pressure is obtained; and
f) monitoring changes in the pressure of the medium to determine the patency of the first fallopian tube.

18. The method of claim 17, wherein the apparatus is oriented using a set of markings located on the housing element.

19. The method of claim 17, further comprising the step of verifying the sealing of the fallopian tube.

20. The method of claim 17, further comprising:
aligning the second injection lumen with a second tubal ostium of a second fallopian tube;
injecting a second fluid medium through the second injection lumen of the inflatable member until a second predetermined pressure is obtained; and
monitoring changes in the pressure of the medium to determine the patency of the second fallopian tube.

21. The method of claim 17, wherein the first predetermined pressure is less than are equal to 200 mm Hg.

22. The method of claim 17, wherein the first predetermined pressure ranges from about 40 mm Hg to about 200 mm Hg.

23. The method of claim 17, further comprising the step of pre-warming the first fluid medium to a temperature ranging from about 15 degrees to about 45 degrees Centigrade before injecting the first fluid medium.

24. An apparatus for selectably accessing a fallopian tube in a patient, said device comprising:
a) a flexible housing element having a distal end and a proximal end;
b) right and left inflatable members disposed on opposed sides of a distal segment of the housing element;
c) at least one inflation line for communicating fluid from an inflation fluid supply to the right and left inflatable members;
d) first and second injection lumens, wherein the first injection lumen terminates on the outer distal surface of the right inflatable member and the second injection lumen terminates on the distal outer surface of the left inflatable member; and
e) an injection means for selectably injecting a fluid into the first or the second injection lumen.

25. The apparatus of claim 24 having a first inflation line for communicating fluid to the right inflatable member and a second inflation line for communication fluid to the left inflatable member.

26. The apparatus of claim 24, further comprising a retractable sleeve reciprocable between a first and second position, wherein the first position is located over a distal portion of the housing element and covers a portion of the deflated right and left inflatable members and the second position is proximal to the first position and releases the right and left inflatable members.

27. The apparatus of claim 24, wherein the inflatable members have a concave region on the outer distal surface of the inflatable members, wherein the first and second injection lumens terminate within the concave regions.

28. The apparatus of claim 24, further comprising a vacuum line traveling from the distal outer surface of each inflatable member through each inflatable member and the housing element to a vacuum source proximal to the housing element.

29. The apparatus of claim 24, wherein a vacuum line surrounds the first and second injection lumens.

30. The apparatus of claim 24, further comprising a biasing means for biasing the right and left inflatable members towards a right and left tubal ostium.

31. An apparatus for selectably accessing a fallopian tube in a patient, said device comprising:
a) a flexible housing element having a distal end and a proximal end;
b) a substantially triangular shaped inflatable member disposed on a distal segment of the housing element, wherein when the inflatable member is inflated the inflatable member has two injection inlets on opposed sides of the inflatable member;
c) an inflation line for inflating the inflatable member;
d) an injection lumen transversing the housing element and a proximal section of the inflatable member, wherein the injection lumen is in fluid communication with the interior of the inflatable member;
e) an expandable plug within the interior of the inflatable member, the expandable plug designed to seal off one of the injection inlets of the inflatable member when the plug is expanded and inserted into the injection inlet;
f) a guide line attached to the expandable plug for maneuvering the plug from one injection inlet to the other injection inlet ; and
g) an injection means for injecting a fluid through the injection lumen and into the interior of the inflatable member and out through the unsealed injection inlet of the inflatable member.

32. The apparatus of claim 31, further comprising a plurality of supporting structures for biasing the inflatable member outward.

33. The apparatus of claim 32, wherein the supporting structures are made of a shape memory metal.

34. The apparatus of claim 32, wherein the supporting structures are made of nitinol.

35. The apparatus of claim 31, further comprising a retractable sleeve reciprocable between a first and second position, wherein the first position is located over a distal portion of the housing element and covers the deflated inflatable member and the second position is proximal to the first position and uncovers the inflatable member.

36. The apparatus of claim 31, wherein the expandable plug is a balloon.

37. The apparatus of claim 36, wherein the guide line is tubular and in fluid communication between an inflation fluid supply and the plug.

38. The apparatus of claim 31, wherein the guide line is made of a shape memory metal.

39. A method of assessing patency of fallopian tubes in a patient comprising:
a) inserting a distal portion of the apparatus of claim 31 into a uterine cavity of the patient;
b) orienting the apparatus with respect to the uterus anatomy;
c) aligning the first injection inlet with a first tubal ostium of a first fallopian tube;
d) inflating the inflatable member to seal the first fallopian tube from the uterine cavity;
e) maneuvering the plug into the second injection inlet;
f) expanding the plug to seal the second injection inlet;
g) injecting a first fluid medium through the injection lumen of the inflatable member until a first predetermined pressure is obtained; and
f) monitoring changes in the pressure of the medium to determine the patency of the first fallopian tube.

40. The method of claim 39, further comprising the step of verifying the sealing of the fallopian tube.

41. A method of assessing patency of fallopian tubes in a patient comprising:
a) inserting a patency assessment device into a uterine cavity of the patient, said patency assessment device comprising a selectably engageable sealing means for sealing a fallopian tube from a uterine cavity and providing an injection means for injecting a fluid into the sealed fallopian tube;
b) orienting the device with respect to the uterus anatomy;
c) aligning the injection means with the fallopian tube;
d) engaging the sealing means to seal the fallopian tube from the uterine cavity;
e) injecting a fluid medium through the injection means of the sealing means until a first predetermined pressure is obtained; and
f) monitoring changes in the pressure of the medium to determine the patency of the fallopian tube.

42. The method of claim 41, further comprising the step of verifying the sealing of the fallopian tube.

43. The method of claim 41, wherein the first predetermined pressure is less than are equal to 200 mm Hg.

44. The method of claim 41, wherein the first predetermined pressure ranges from about 40 mm Hg to about 200 mm Hg.

45. The method of claim 41, further comprising the step of pre-warming the fluid medium to a temperature ranging from about 15 degrees to about 45 degrees Centigrade before injecting the fluid medium.
